(19) United States
(12) Patent Application Publication
(10) Pub. No.: US 2010/0076530 A1
(43) Pub. Date: Mar. 25, 2010

(54) CONDITIONING GARMENTS

(75) Inventor: Alan Paul Oliver, Rhyl (GB)

Correspondence Address: Workman Nydegger;1000 Eagle Gate Tower, 60 East South Temple, Salt Lake City, UT, 84111, US

(73) Assignee: SURVITEC GROUP LIMITED, Birkenhead, Merseyside (GB)

(21) Appl. No.: 12/442,364

(22) PCT Filed: Sep. 11, 2007
(86) PCT No.: PCT/GB07/03433

§ 371 Date: Mar. 20, 2009

(30) Foreign Application Priority Data
Sep. 21, 2006 (GB) 0618629.0

(51) Int. Cl. A61F 7/00

(52) U.S. Cl. 607/104; 607/112

(57) ABSTRACT

A conditioning garment has an array of tubes (11) for passing heated/cooled fluid to heat/cool a wearer of the garment. The array of tubes (11) terminate at a connector (16) for connection to a source of heated/cooled liquid. The connector (16) comprises a male part (15) and a female part (17) with a releasable latch (22, 34, 40) acting between the male and female parts (15, 17) to hold the male part (15) in engagement with the female part (17). The releasable latch includes a release member (40) moveable in a rectilinear path away from the connector (16) to release the latch and allow separation of the male part (15) from the female part (17). The male and female parts (15, 17) are self-sealing on disengagement. This allows for automatic separation of the male and female parts (15, 17) in an emergency.

The invention relates to conditioning garments.

A known conditioning garment comprises at least one tube for passing heated/cooled fluid to heat/cool a wearer of the garment. The at least one tube terminates in a connector for connection to a source of heated/cooled liquid and the connector comprises a male part and a female part. A releasable latch acts between the male part and female parts to hold the male part in engagement with the female part.

Such conditioning garments are often used in situations where an emergency exit is required. For example, such conditioning garments may be used in aircraft which may require emergency exit using an ejector seat or a tank where emergency exit may be required if the tank is damaged. In these cases, it has been customary to shear the connection between the conditioning garment and the source of heated/cooled liquid in order to allow rapid exit. This is, however, unsatisfactory as the shearing action releases the fluid and may leave a considerable length of tube to impede the wearer.

According to the invention, there is provided a conditioning garment comprising at least one tube passing heated/cooled fluid to heat/cool a wearer of the garment, the at least one tube terminating in a connector for connection to a source of heated/cooled liquid, the connector comprising a male part and a female part, a releasable latch acting between the male and female parts to hold the male part in engagement with the female part, a releasable latch including a release member moveable in a rectilinear path away from the connector to release the latch and allow separation of the male part and the female part, the male and female parts being self-sealing on disengagement.

By having a release member moveable in a rectilinear path away from the female part, the release member can be made to release in emergency situations and so allow a rapid exit without damaging the system.

The following is a more detailed description of an embodiment of the invention, by way of example, reference being made to the accompanying drawings in which:—

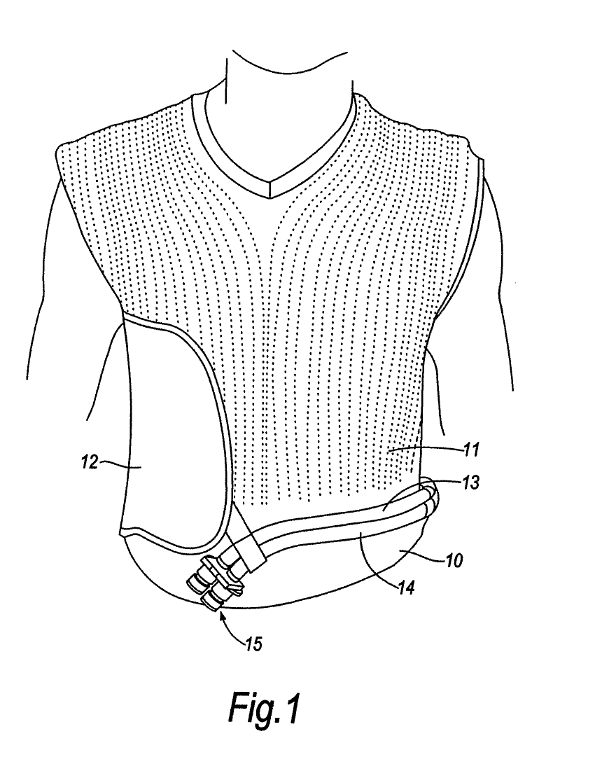

FIG. 1 is a front elevation of a conditioning garment including a male part of a connector,

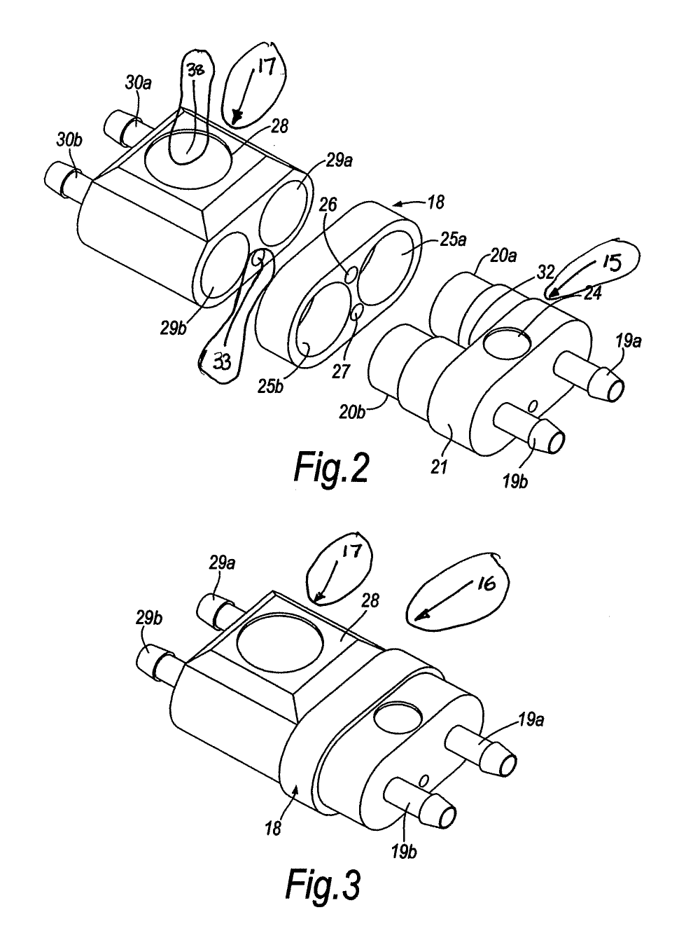

FIG. 2 is an exploded view of the connector, including the male part of FIG. 1,

FIG. 3 is a view of the connector assembled,

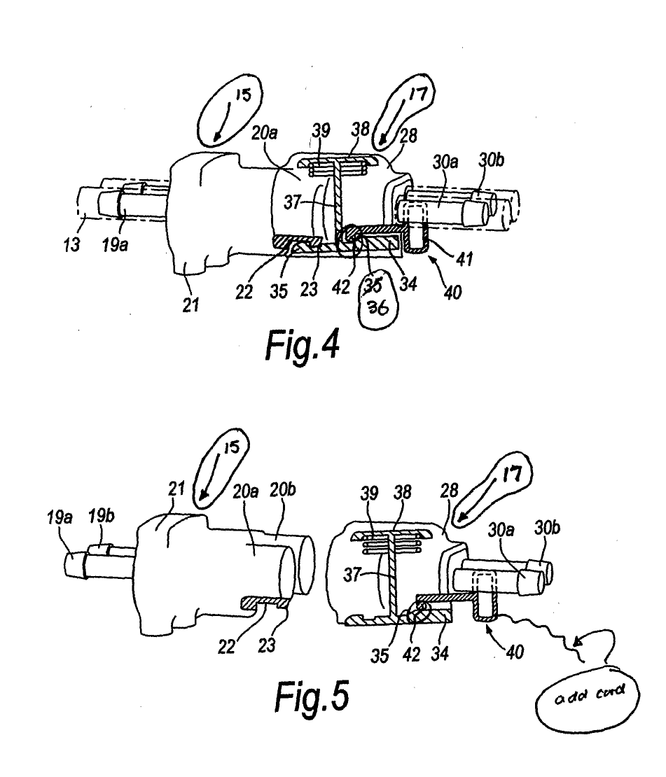

FIG. 4 is a side elevation partly in section of the connector of FIG. 3 showing a release member and a latch, and

FIG. 5 is a similar view to FIG. 4 but with the male part disengaged from the female part.

Referring first to FIG. 1, the conditioning garment comprises a vest-like backing material 10 and an array of tubes 11 for passing heated/cooled fluid over the vest 10. The vest 10 is open at one side and, at that side, a side flap 12 is provided to allow adjustment of the size of the vest 10.

The tubes 11 extend between an inlet manifold (not shown) and an outlet manifold (not shown). The inlet manifold is connected to an inlet tube 13 and the outlet manifold is connected to an outlet tube 14. The inlet tube 13 and the outlet tube 14 are connected to a male part 15 of a connector 16.

The connector 16 is shown in more detail in FIGS. 2 and 3. As seen in those Figures, the connector 16 also includes a female part 17 and a bridge piece 18.

Referring to FIGS. 2 and 3, the male part 15 comprises first and second parallel pipes 19a, 19b carried by a housing 21. The end of the inlet tube 13 is pushed over the first pipe 19a and the end of the outlet tube 14 is pushed over the second pipe 19b. The pipes 19a, 19b lead to respective passages 20a, 20b carried by the housing 21. As seen in FIG. 2, the passages 20a, 20b extend side-by-side but parallel from the opposite side of the housing 21 to the pipes 19a, 19b. The male part 15 also includes a first latch part 22 that projects from the housing 21 in a direction parallel to the axis of the passages 20a, 20b. The first latch part 22 is formed by a flat elongate member carrying a triangular section head 23 at its free end. The latch member 22 can be depressed by pushing on a button 24 provided on the housing 21. The purpose of this first latch part 22 will be described below.

The bridge piece 18 comprises a mounting formed with a pair of parallel side-by-side holes 25a, 25b for receiving respective passages 20a, 20b on the male part 15. In addition, between the holes 25a, 25b, the bridge piece 18 is provided with a latching aperture 26 and a second aperture 27. The latching aperture 26 is for engagement with first latch part 22 in a manner to be described below and the function of the second aperture 27 will also be described below.

The bridge piece 18 can be connected to the garment so that, when engaged with the male part 15, the male part 15, and consequently the female part 17 when engaged with the male part 15, are located relative to the garment.

A female part 17 comprises a housing 28 formed with a pair of side-by-side passages 29a, 29b. Each passage 29a, 29b leads to a respective pipe 30a, 30b projecting from the housing 28. A supply tube (not shown) leading from a source of heated/cooled fluid (not shown) has an end pushed over the first pipe 30a and a return tube (not shown) leading from the supply of heated/cooled fluid (not shown) has an end pushed over the second pipe 30b.

FIGS. 4 and 5 show the male and female parts 15, 17 with the bridge piece 18 omitted for clarity. The length of the first latch part 22 is sufficient to allow it to pass through the second aperture 27 in the bridge piece 18 and enter the aperture 33 in the female part 17, as the parts are fitted together as described below.

The female part 17 includes a second latch part 34 of the latch member. The second part 34 comprises an elongate member extending parallel to and between the passages 29a, 29b in the female housing 28. At its end opposite the pipes 30a, 30b, the second latch part 34 is located in the female housing aperture 33 and is provided with a triangular section head 35 complimentary to the head 23 on the first part 22 of the latch member.

A portion of the second latch part 34 remote from the head 35 is provided with a ramp 36 for a purpose to be described below. A connecting bar 37 is fixed to the second part 34 and extends normal to the second part 34 through the female housing 28 to connect to a push button 38. The push button 38 is urged out of the female housing 28 by a spring 39 acting between the push button 38 and the female housing 28. Depression of the push button 38 thus moves the second latch part 34 against the action of the spring 39 in a direction normal to the length of the second latch part 34.

The female housing 28 also carries a release member 40. The release member 40 extends parallel to but spaced from the second latch part 34 and has an end projecting from the female housing 28 on the same side as the pipes 30a, 30b and terminating in a loop 41. The opposite end of the release member carries a follower 42 that engages the ramp 36 on the second latch part 34.

The second latch member operates as follows. The male part 15 and the female part 17 are aligned as shown in FIG. 5 with the male passages 20a, 20b in alignment with the female passages 29a, 29b. The male passages 20a, 20b are then pushed through the bridge piece holes 25a, 25b (not shown in FIGS. 4 and 5) until the male passages 20a, 20b enter the female passages 29a, 29b. As this happens, the end of the first latch part 22 enters the aperture 33 in the female housing 28 and the head 23 engages the head 35 of the second latch part 34. This moves the second latch part 34 against the action of the spring 39 to allow the head 23 on the first latch part pass the head 35 on the second latch part. When the male passages 20a, 20b are fully inserted into the female passages 29a, 29b, the head 23 on the first part 22 engages behind the head 35 on the second part 34 so preventing the male part 15 being disengaged from the female part 17.

The male part 15 can be disengaged from the female part 17 in two ways. First, the push button 38 can be depressed to move the second latch part 34 downwardly, as seen in FIGS. 4 and 5, to disengage the head 35 on the second latch part 34 from the head 23 on the first latch part 22. The male passages 20a, 20b can then be withdrawn from the female passages 29a, 29b. Both the male passages 20a, 20b and the female passages 29a, 29b are self-sealing in known manner.

Secondly, the release member 40 can be moved in a rectilinear direction away from the female part 17 of the connector 16. This moves the follower 42 along the ramp 36 which moves the second latch part 34 downwardly, as seen in FIG. 5, against the action of spring 39. This has the effect described above of disengaging the head 35 on the second latch part 34 from the head 23 on the first latch part 22 so allowing the male part 15 to be disengaged from the female part 17.

The conditioning garment described above with reference to the drawings is usually used in military transportation such as a tank or an aircraft. The transportation will be provided with a heating/cooling system with a supply pipe and a return pipe connected to the female part 17, as described above. The loop 41 on the release member 40 is connected to one end of a cord. The other end of the cord can be connected to a fixed point in the transportation system with the cord being shorter than the length of the supply tube and return tube.

In the event of an emergency, such as a tank being hit by enemy fire or the use of an ejector seat in an aircraft, movement of the person away from a normal working position will tension the cord which in turn will move the release member and so allow disengagement of the male part 15 from the female part 17 without any action by the wearer—the movement of the wearer will simply pull the male part 15 away from the female part 17. Thus no liquid is spilled, because the passages 20a, 20b, 29a, 29b are self-sealing and the wearer is not impeded by trailing supply and return tubes.

It will be appreciated that there are a number of modifications that can be made to the conditioning garment described above. There need not be an array of tubes on the vest; there could be a single tube. The garment need not be a vest; it could be any garment. Although the connector 16 is shown with two tubes; it could have one tube or three or more tubes.

The second latch need not be as described above; it could take any suitable form. The push button 38 is optional. The latch could be a pivoting latch that is rotated by operation of the release member 40.

1. A conditioning garment comprising at least one tube for passing heated/cooled fluid to heat/cool a wearer of the garment, the at least one tube terminating in a connector for connection to a source of heated/cooled liquid, the connector comprising a male part and a female part, a releasable latch acting between the male and female parts to hold the male part in engagement with the female part, the releasable latch including a release member movable in a rectilinear path away from the connector to release the latch and allow separation of the male part from the female part, the male and female parts being self-sealing on disengagement.

2. A garment according to claim 1 wherein the male part defines a fluid passage through co-axial with a fluid passage in the female part when the male part is engaged with the female part, the rectilinear movement of the release member being in a direction parallel to the common axis of said fluid passages.

3. A garment according to claim 2 wherein the releasable latch comprises a first part on the male part and a second part on the female part, the first and second parts inter-engaging when the male part is received in the female part, said movement of the release member disengaging the first and second parts to allow the male part to be removed from the female part.

4. A garment according to claim 3 wherein the release member is mounted on the female part, the release member moving the second latch part to disengage the second latch part from the first latch part and allow the male part to be withdrawn from the female part.

5. A garment according to claim 4 wherein the second latch part is spring loaded into engagement with the first latch part, the rectilinear movement of the release member away from the female part moving the second latch part against said spring loading to disengage the second latch part from the first latch part.

6. A garment according to claim 5 wherein the second latch part is elongate and extends in a first direction parallel to said fluid passage axis and the direction of rectilinear movement of said release member, the second latch part being movable in a second direction transverse to said first direction against said spring loading.

7. A garment according to claim 6 wherein the movement of said second direction is caused by a follower acting on a ramp surface as the release member is moved away from the female part.

8. A garment according to claim 7 wherein the follower is formed on the release member and the ramp on the second latch part.

9. A garment according to claim 5 wherein the first latch part is flexible so that, as the male part enters the female part, a head on the first latch part engages a head on the second latch part to flex the first latch part, the head on the first latch part pushing the head on the second latch part and engaging behind said head when the male part is fully inserted into the female part.

10. A garment according to claim 4 wherein the female part includes a push-button, depression of the button disengaging the second latch part from the first latch part.

11. A garment according to claim 10 wherein the second latch part is spring loaded into engagement with the first latch part, the rectilinear movement of the release member away from the female part moving the second latch part against said spring loading to disengage the second latch part from the first latch part, wherein the second latch part is elongate and extends in a first direction parallel to said fluid passage axis and the direction of rectilinear movement of said release member, the second latch part being movable in a second direction transverse to said first direction against said spring loading, and wherein said push button is connected to the second latch part by a rod extending in said second direction, depression of the push-button moving the rod in said second direction to disengage the second latch part from the first latch part.

12. A garment according to claim 11 wherein the push-button is spring-loaded to provide said spring loading on the second latch part.

13. A garment according to claim 1 wherein the release member is connected to a cord, tension in the cord moving the release member in said rectilinear path to allow the male part to be removed from the female part.

14. A garment according to claim 1 and including a bridge piece for connection to a flexible material for mounting said at least one tube, the bridge piece including a hole through which the male part extends before entering the female part for mounting the connector on the material.

15. A garment according to claim 14 wherein the male part includes an additional releasable latch member engaging the bridge piece to connect the male part to the bridge piece.

16. A garment according to claim 1 wherein the male part includes two side-by-side but spaced fluid passages and the female part includes two corresponding side-by-side but spaced fluid passages, each passage on the male part being received in a respective fluid passage of the female part.
